SEQUENCE LISTING (1 sequence)

SEQ ID NO: 1 - DNA, 33991 bp, Bacteriophage Str-SUP-1
taattgattgtagaataaaaaaaagccctacgctcaaattttggttgaggagagcgtaaggcggatcatgtatagtaaaaacctgctttacagtaggtctctttactatacccattttaacaaaaaatgaggggaatgacaatgaaaattaaaccatatataaaaaacgggaaaacgtattacaaattcgtactatatgttggtgtagttaacggaaaacgaaaatacattaagcgtgccaactttaaaacaaaggccgatgccagagcagcaatactttctttgcaagaagaaattgaccgacctgtaggagatatgaccttccaagagctgacagagaaatggctaaaaatctacgaaaatgaagtcgcagaaagcacctatatcaaaacaagtaggaacatcaaacaccacatcactccaactatcggacacaggaggatttctgagattacagccctggagctacaaagccatacacagcaatggtgttccaaattaaaatacggtaggaagatattaggcttagtcaaaacgatatatcgttatgcagtacgtatgggcttcattgccattagtccagcagaccgctgggttgaaaatactattttacattgccccacacttctgtggtctggccatttttacgaaccttaatcggcatccaacgacgaatacctgtgctagctgaaatccaactaatccatacgtagccgttataatcaacacggacacggtcataagtcactgactctcctttgtagtacatgccttgtgtacgaccgtttaagttcggctcattgcggaaatagatgtcttctgtggcagtaaacacacccgtttccgcatattcacgggcaccagtgttaacactagccgttctaggttgggtagttgctactttaggggctggctgtggtgctactgctttgccttgcaattgcaatagataagcctgtactttacctttaaaaccgtcccagttgccattatccaagatgcggtgtgggcaatatttgccagaccaatcttgatgtttgcggatgcggtcaatgccccagcctaactgatgcaagataatcgctgctaatttagctccgttatcctcagcttttgcatatttaggatgtcccggagtcagactataacaaatctcaatgccgattgatttacgattacctgtcccatttgtgccgtctccacaatgccaggcattacgattgtgcggaatggcttggatggcctgctcatcatccaccgcccaatgataagatacttcgttccagttgccattcatgtaactgatttccgctaaagcagacgcattgtttgcggtattgtggatggtcaaccattgagggaccatagcgtttggacacttaatgccatacttagtagcaggcacgggcatttgaatcattttaagtcctagattactcatgttctactcctttccgtccaaatcatctatttctggaaattcgtgatcggcgatatttttttgatagagttcgtcgttgaactcgattacttcgtcaaattgcatgattagtcctccttttcaaaattaaatgcaatattcccctcgcccaaatctactttcatttcttcgggctggatagtcccagtagaaattttgctggcatcaagggctttaggttcgcttttaactaatttttcaagcgctaaaatcacattagcaaatatacttgtatcgccttttgctttaccgtagttttctaccaagctcttaaatgtcaaaataagatacccaacgtaaattgtgtaaagaaatgccacacccgtctgctcaggcaaaagcaccgacattggaattaagactgtaagcaagataatccccatcatcttgcgaataagaccgttgatgccaatttttgacttgtactcaatgtcaggattaatcatagcagcaaaggtacccgacagaaaatcaacgatttccatgactacgattaaactaagcaaaaagaggacaagaccgtcctccgttgcaataagttcgcgtaggaaatcaaacattcctgcaggatgcggtggcattttagataacatcataaccattctttacgcctccgctcctgctgtcgggtctgtccagtctggatttccgttttcatcaaatttcataatccaatactcttcgttgagcatgtcagcaatattaattgtcgcagcggtaccaccccactgattaaatgcccaaatcgtttccacgtcgacaaacttgcgacgaccgtttacgattgctggacgtttctgcacgtcacggtacatatagaagtcttgtgtcgctgacttacaacggataaattcaccattctctttcatgtagtgaagcgcagtcgcaaggtcaaatggttgtgttgtttcttccaaattaagcaatgtgttatctgtagtttgagtcatgattattctccttcgatgatttcgtccgctggtttagtagcttcgtccagttgctgagtcaaatctgcaatctctgcttgcagttgcgtgataatctgctgtgcttctgtcagctgtacagctaacagattcttagttgtcatctcctctgacaattttgccacgaggtcgttattagtcaagcgtagagcttgcgtgatttggtcttggttcatgttgtctcctatccaatattatacgtattatattcgttgataaccgatctgcgcatagccatatcatcaggattccatccggtaccattccaatgtagccaacacctccacagagcacggattgaattgataacatcgactagttcataagaggtagacaattttgtggctgtaatcgttataccgttaccattaaaaccatggccgagataaattttatctccataaatttctgtttggtccgttgtatcattagaacgaaaaatccgaatacctgcaaatcgaccggtgttttgagatttgaccccctcattgtgcgaggtgacacctaatccgacatatgtccctcctgccgtagcgttgttaaaatgcaagaagcctgtaacatcgccaaccttacgaataagggcattgcctgaactattgaattcaatggtcgcactggaattaaaggcaagtttagcattattgagatcgaattctgcagcaccatttgtcgctttaagtacacctcctacaatttgaccggcagacatagttactgcttgaacactcgtgataaaggcactttgagcaaacaactgcttaaacaaagcctcgtttgctgtcatcttgttgacaaaagcttggtcaaataccatgtgactgcccgtgatgctattggtagcaatcctagccgacgctagatagcctgttgtgattttgcctgcgtccaaattgccgatcatcgcgttcttgattacgccgttatcaatcaaagtctgacccgtgatatgcgtcagtcgaccatcgattctattagtaccattagccagtaaattgatttggttaagaactgtaccagcactagtcagatttttaatagcgtacgaattattaagctgactaacctgtgtagctgtaccgctaatcttatccttgacctcggtcatgaacaagctgtcagccatgaccatacgagcaactctgtccttgatgccagcttccgttgagccaatgatacgctcatagagctgtgaggtctcctgcacccgctgaaacgcaacttggctagccttactgtcaacagaggtctgcatcgtcgcaaaacggccatcaaccgtagtacgatactcagcgattttggtctctgtgtaggcctgttggtcttcaggggctggtgaccagtcggtatgcaatgtgccatattcaaccttaatcttcgtttttaagtctgttccaatagctaaattagagtaccctattccgtacctcaaagtgataaaagctactcctgatagctgggacggagttagactaaatttattagtaaaatgtcccgtgaatgtttttagcgtattagaatttgaatcaaaataggatacgtaatttttggtaaacctatcattagttatttggacaaggattgagcgaccaggtattactgggatttttatcccataaaccgggcgatagggattagggtgagcgacagcatgaccgacaaaggcatcgactccagtggttctgaaggaaatctcaccagtagaaacattttgattgaagttcgtaagttgcgtcgttgtaatttgagaattagctgacaaagcatatatgttacttccaccaacttgcaaactttcaaaccgctgtgtcacacccctagcatcctcctcgtatttagccttagccacatagccgtccgtgactgccgtacgcagagcagttacttgccttgctgtctcgtctcgactagcggtaaagtactgactagctcgtgtgccttcagcgttcttatagctttccaagctctctagtcgagtatcgattgcagttgcagtttgctgagcgtaggccttagcatctacagctttaccgtctactgtttcaatccgtctagatagctccgcgctgttttcagacgcgttgcggatatacgttccaacctcagagcgtagcgtttcaactttaccttcggtatcctcaacagctggacctgtgtccattggtttggttccccgagataattcaacttttctaaaggacactgagcctatttcgctatagcctaaaataattctccgatgtccaaagtcagagtttggtttaactgtgccagtaacagagtatctgacccattcagatgtggcagatattacatttctgctgcctcctaaatgggttacaaccccgttgtaattcgacagtccattatccctaagttccaaccaaaatttcggacttcctgaattagctttgacctcgacagataatgtataagtttctccgacttctaattctaagagagcagtcgagtccttctttccaccaagtctaccaccactctccgagtgaatttgtaattggtgccacgtattagtagtagttgtgagcgtatagataccgttatctgtacttctatcggtatctgaggaatcaccttgcgaatactcccacaaaccacgactaaaatcgtagtcctctgcatagtttctagcaccaaccctcagattctcgaaccgctccgtcacaccatcgattccactctgcaagtcagcagtcttacgattgatattgtcaatctgacctgtctgcgtgttgacagtctgcgtcagagcttcgtattgcgtcctcgtttggctcagagtgtcttctacggtctttgtccgactggtaacactcgtgatgtcgccagtagccttagaaactgttttagagagttctgagacggtcatcttagtgctattagcggtgatttcaacacttgctactcgattggtcaattccgtctgtgtttgtgcctgagcaagtatagtttgcgcctgagacataacatttgtccgaattgtaccaatagaccgctctaattcgagcgctttggtattagcttgaccaatggattgattaaatgcgtttcgtacccgcattaaatctgttacagtttgttcagctatttctttagccgaagtagccttagctaatacatctgctatttgacggtcttgggcttgctgggcaagttgatattgacggtccgattcggcgagctgtccatctatctgttgcttgatgttatctgcatgggcttctgcttctgcttttgactgctcgatgctgtcgttgatttcctcgactcgcttttcaaattcagtgtcgaaatggcggttagcattatcaatttccttctgcagtttctgttcaaaggatgctgacaatctaccggatgctttgtcaacactgccagcgaccatgcttgcgaccgtcgagccaaatgtctgcgataccttaccaaatcctatggatttcaatcgcttggacatcggtgcaaaatggtagcttgtgattttcaaacgcaagtccacatcaaatctctcatggaatacacttactgtgtcgaacatcttaacagatacatcagacttgcctttgacatccagattgatagagttttctaccaaatcacaaagagttgagccaaaatacctctgcccataagctaaaagacttgcttcatcggttacatcttggtcattgacctcaatatctgcttcgaagatttgacgatattgcccaatcaacggactgtccactgtcacagcgattacacggtcttcctcaccctcagactgtccttggattgtctttttgaggtgcaaacgtgtctttaaattgttaatgttttcagattcttcgtagctactgagattctttttgtacataaacagcgactcattctcaataccgccgttttttaacaatttaacttggtacttgtcacgcaccaagtcgccaccccactgaccgacaatagagtgtttttcttttaccaaagcatccattacagatacattggactcattaaacgtatgacggtctaaaatgtcactaaaaaacgtaaatggacagttccgcttgatactaccagctaaggcagtcatagccgtctgcccaggtaccctgtcaaccgagatagagttgatagagtagtaattcaatagagttgcaacctgcttagcatagacctgcacatagccgttggctttctttacttcaaaaatgaaaaactcttgctcgccgtgcagatcatcggccagcagaaagacttcccttttgagtagtttccacttcccatcagtcaatggaaatttaaaagaaagctggtaggtactattggcttcctgcacgatgtcatcatcataagcaagattgagcgggatgttcccgtctttaagatagatcaaaccttgtacctccaattccctttgatagtaatttttgtgactgttccacttgttgaaataccgcttcttccgacagggatttcaaaaaacggaccacgcttgcgaatggtattcttcacagcaccattcttgtcatagatattttgtcgcttatgccgacagtcgatggtcgcacgagtatttagttccagttccatgacctgctgaccaatagtcaaggtcacccgtcccgaaccctctatgatgatgatgggctcagaatagaccgtacctggattttgaatagaaccagatgaagccaaaacgacatcgcccggatttttgatatagcgaaacggatgcatggacacagtaatttctaccttccaacgatgtggaccaagtggcacgtagctactagataccaaatcacagtagaaaaggctatccctcagatagccgaattccaccgtattattttccgactgaaaaacctcaatcagtcgcatagcatctgccaaatgtcgtagcgtgatgataaatgtcctgttgtatccatcatacgcaccctcgctgacatgtaattgaccattggcaccgaagacctcgacttgttctgaccgttcaaccgaagtttgtgctttgccaaagtcaagaacgtggcaatctgccaaggtcgaagtatctaagccgttgacgattatatagttcattagataccctcccttgccataatggcaccttgatgcatgtagctattttgtgccattttctcaccgtccaaatagaccgccaactctttatcaaccaaagcagacaagaagcgctcgatattggtcagacgagatgccaaactatttccaccatcacttctagccaaatcgccagtagaagacagcaagtttcgacgcacatcaaccgatgcacttgaagtcatatcataggccaaagattgattttcaaaaggcttggcaatcgctccagccatgccagataccgttcccatgacatccctaaaaccaactttcaagttcgtatttaagccttgcatgatagcaagaccagcaggtttcaaaagtactcggtcataagagataggccccttgttttcagcaatccaatcagcaatacctccgacaaaacttgttacttctccccacatggagcgtaatccgtcaacgaaaccgcttaaaattgcagcaccagcatcaaataggtcgatgtcctcaacaccgcctagcaacttagcaccaatatcgagtgctgcttgaccaacattccctaaaatgctcagaattccactgattagagttgttaaaatttccacaccagcagacaatatatctggtaatttcgataaaagcatcgataaaaactctcccgcaagctctaacgctgtggaagtgagttggggtaattcttgaactaatccattcacaatattgacgacaaattcaacacccatccgtaaaatatctggaagattttgtgtcaaaaaatctataaacattccgaccaattcaatcgctgcactaccgatttcaggcaaacgctcaacaatgccctgcaccaaattaacaagaatttcggaacctttttccaacatttgaggcaacgagccttgaataacttccgtaaaagacctaaaaatttcctgcgccgataacagcaagtttggaatatttaagataattccatcaatgagatttaaaagcaagtcagaacccgcttgtaacatcgtaggtaaaaagttcgcaaatgcaacaacaagatcaagaaaaatatagctaaatgttatcaacaggtctggaatagcactgataatcccttgtccgagattagaaagtaattctacgcctgtttgcataagtgttggaaactcttcgataaaccactgacgaacgttcgctgtcagacctgtaaccaacatttcaaactctggtaaccctgtctcaactccaatcacaagaccagacaacagtgcctttccttgctccaaaatcagaggcattgcctcagcgataaacgttccaattgcagatggcagacctttgaagatattaccaaccatgggaaagaaatttccaaataagaaattagaagtcgtcgttgccaaggcttgtaattgtggagctatattctcgcccaaggacaagcctgccagtgtattttcccaactcgctttcatagcagctaaggaccctgaataagtattctgagcttcggcagctgcaactccagctatccccatgctttcttgcacaaggtggatagcttcgacaacatccgcatagttgctgatgtcaaatttacgccccatcgcagacggcaaaccttcagcagtcttcaataatcgttccatttctgactttgtgccaccgaagccgagcttaagattatccagcatagcatagtttccgcgagaaagactttgataagtctgttgtatgacgccaatatccgttcccattttcgcagcgttgtctgtcatgtccatgatagctttgttagccatgttaatcgctttggtcgaatcaccaccaagtgattgttttaagctcgcacccatggatacagcttgctctgcgtatgtatttgcagaaacacctgccttgtaagcttcttgcgcaaacctcttagcagattcttgtgcaccatcatagatagtatccagaccaccgaaagattgttgcaaatcagcaccagcactcaaagcagaaccaattattttcccgattcctgcagccgcaagtgcaccgcttaaagcagagacgagcgaggctccaaggcttgacccagctgacctaccagcgctatcaacttcaccacccaataattttgatattgaaccgctaatgccttttgcggacgggactatctgcacataagcagaacctaaatcagtcgccatgctcctcacctccaatctctatttctagcgcacgcatagcacgttcaaactcctcaccagaagaaaagacacgttcctcacgttccactttcgacccttccaaggcttgagtgacagagtttggacgattccgtccagcctgcccgtccttagttttagcccaaaagagcaaacgaaccgtatcatagatacctgcaagcagcagagtatccaagtcttccttctgaccagacaagaccttcttaatccgtgatttttcacttaacccgcaagcaaaaacagctacccgagttataggtagctgtcgataatcatatatgccataggtttcagccaaatcacagacaagagcatcctcatctactgcgatcattctggcgaggattgcgagttttttaagtctttgacttgttcaaagacatccttgatttcagcaccaagagcagaaataggcaccagccctttttctgtacggacatgctccttgagtttcttcgcttcgtcaccgagcaacaaattgacaatccgaatcatggcaccactgtctccagattcttcagcagcgattgcctcgaaaagctcatagttttccaaacggtcttggtcgatgctaagcactagacctgtactagtttttacttcaaacattcaaacctccttacgatgacgatggcgagcttgttcctgttgactcgctagctcccttaatgtactcatagtgtgtattgcccttgttatctggcagagcttggatagtcgtttcatacccagccaaatcactatcagcgtaggtaatctccccgacttccagcaccttagcattcgggataacaatccgcttcttcgcaccatttttcagcaacatgtcaaccaccaatggatgcacaggcaattccttagagttgacctctaccgtgatgccagttgcaagttctcctgtcacattttcagggccgtagacctctttcaagacatccacgttcaagacttcaatcagcgtaaatgtaaatttgtctgattttcctgtctgtggagtgtcgaccacatcgccgccccatgctttcagttcttccgattcacgagtgtcctcgttggtcaacccatcctctgaaatgtagcctaagtttttaaaagccgtgtttagcttagtagtcgcattggtcggcaagctcgttccgattggagctgaggaaatagctcctgcaatgtctggctttgctgacgacacaagttttgcatctgccattcaattttctccttttcaaaaataaataaaatcaaccaccgcttgatagcgatagcgtttagtttccgtatctgtaaaattatagtccgagttcaattcaaccctgctgataaacggcaattctaccatcttctcaacagcttgtttgacctcttcattcaaaatggcagcttcttccaaagactgcccataagattgtatcgccaaagtcgcttgattcagatggttctttttgccaccgctcgttttctcaaaaatcacaaaacgctcaggcatttccttctgatgttccgtatagacaggcacagaaagatgctcggttaaaaagttcaacgtgatgacttcaatcatgacttcaccgccttcagcaaggtattgtgcttcttgttatcccgtttagcttgatagctatcagcatagaccatagcgttggcacgagtcttaccgacatagatatccgattcatacccatcgcctgcacggtctcggatagcatttgccttgtcagtcaaaactgcctgcatctcaggcgatttcaacaagtcacggacaccagcacgggtcaacttgaatttcattctagccataccgttccaccatcactttcttgttccactcgagcgggatcagatgttcaagaccttccagcggttcaccaactgttctccatcgttggccaaaaaatcgaacttccttgttcgtccagttgtacatatcccctttaggaatagcgagagtatagtcaactttttttccggtcagattgatttggttggtaatatcctcagttgatgcaggagcaatcaagacattctcaaccacaatctccacgtctgcagtcttgggatgaccaaacgaatccttgccaatgatctgtttgtcaatcaagacaattggtatacctttaatccgtcccataaatatccctcgctccaaaacgttgtttcttaagccccaatcgcttcagctcgctatctttgataaagagccctccaccagggaccaaaaaagaacctgacaccgaataaccaagagctgactcgttaaattgagtcataggctcttggtctgttgatgtcatgagagtgcgagcgaccacatctaccgtgacggatttgacaacagaagcaaagaccaagctgtcactggtcatgctatccaaatccttgccaactttctgggcctcatgtcgcagagagtctgacacaatctcaagcaaggcttctgcccgctccatttcgtcaaacttcaacggtctccacaaggtctccaagtcagacgtagttgcaaaaggagtcataggctactcctcttcattcagattctggctttcttggtaaagttgcaacaactcatctttattcgctttcggattaaattcaatccccaattcggtcaacatcgacttcaattcaggcactgtcggagttttcagaacatccttttctttaactagctcccagttgccttttagttcgctatctgtgacaacgattgctccagtttctttatgtttataaatagccatagggcacctcctaagcttgttcgacacgagcaaaggcagtctcatccaagataccccaaccgataaatgcctctgtacgtagcaaaatctcattgtaagctttcaaatcacgacctgagccatctggatcaccatattcaataatttccattgggatgttttcagcatacccccacttgaatttattcttaaagtctccaacaatggcatggtcagtcttggcagtaccgccagtcacagttagcgtcttgttgatgtcgagttccatgttgaagaaattgtttggtcgttgaccaaagcggaattctgggtacataataccatcaaatttatccttgattttagacatagcctgacctgcaactggggacattgcaataccagtcgcttcatttccgttaatgacaatagtttgaacagctgcatcaatgttatcatcgattttatctgcactataggtaataacattccctgtaactacaccatcaaacgaattagtcgctttaaacgatgcatctgttaaagattttggttcaagaccatgtaaagcagcgatgtcaaaagcctctgccatctttttggcaaatccatcggcatagtgtttcaaaaaatccaactgcttctcttctgatgcatatttaaattcatcagtcaaacgggcttgatagacaaacttaagtggtttgattacttttgatgtgaccgtagctgaaccagctcctttttgctccccttcaccaacaatctgtgcattaccatccagattaaagataaattgctcagtaccattgaatggtattggagtttgagtcgatagtttagcaagagtggaatgaccttgaaccttcgacataatttcttttactaattctggtttaaaaagtgttccagctttcatagatgttgacataaattattctcctttatttaaatcttttataacgtgacgccatgcagcatcctcgccttttaattctggctcaatgcttttgagtggtggaattggatcttgtggtttcataaggcttgcaaagcgttccgcatctgccgtaaggcttgcttcatcttcgccttgtaaacgatcagcaaattcaataggcaagccatatttgagagcaatagccgtcttcgttttcgcagtttcatagcctgcaatagttgacttatacccttcaatttggctttcgaaatcagctgcagtctgatttgattgtgcgaccgtttctttgagcccagcgttctccgcttccaatgttgaaacacgagatttcagttcgtcgtagtcagcatattgctctttcagtcgattcaaacgagccttgataatggtatctagctcttcctgtgtttcgattgctttaaattcagacatatcaatgtctcctttctcctgctttcccggcagttcggtaatattttttgcattaaaaaagacaggtcacccctgtccacttaatacctaatttgttgctttttcttaggcttggttgtagcacaagcccaatgtgccagcaaagcactgtccattaaactaatatccatatcgtcaaagtgtgagcgatagccaaaaccaccgtttgagccgatattccgcttgtcgcagttagttacgaccttcgacagtgacggttgcccattgtgacaaatcgtgtgctggtagatgccctgttcccacattgagttagcgatgatgatttccttgacggttggcaagatgacattgcggattttagagtctttcaactcctcttctaacatcttctggccactagctccgtcaatgacgatttgagccacatctgcctgtttcaaaaaggcaaccagccaggcactaccattccgcacagattgacaatcaatggtttccacaaagaattgaccgtcttctgtccgaacagcaatagacaaagcaacgttggtaccgtcttgcccgaacttgatgccagcaaatagcttgccagtcagcttcggcatatcgtcaatcttgagtgcgttccactcggtctcagatatagctgatttctgattgtaagtcggccaaaaacccaaacgctgcacattatggtctaacttatcctcgccaagctcagcctcaatcttacgttcgttcaaatgatagcccatggacggattagagttataccatgctgctacatcgtcaatttctttctcctgatccacagaccattcagcccatcccgaatacttccccttgccaaacagacaagtttcacgatacttagtaaagaccgtaccgcttgaaactggagtcggtggtgttccacacatgaccgtcatcggattgtccgaatccgtcacagtatacttaagagccgattcctgctccgtggtgtattcctgggcttcgtcaatgataagcaggtcaaatccctcaccaagaccgccagtacctgttcgagtacggaactggaccacaccgcctgtctcatagagctcgattctttcctgccccttagctcgaatggagttaaagtcctctccgtccacatagcccattttctccagatatcgcttgaccttttcgaaggacgagtgcgatgttgaaatgcggtgggctgtgtgcaggatattgagaccgttgtgcaagccccaaatctctagcatatagagaatttcagacttaccattccgacgcgggatagagtaaccaaacttttgatgtacccacagaccattcttgtcaacagccatgataggattgagcaaattgacctgccaagcatagcaagacagacctgttcgctcatagagttctatcgcttctttggctttggaatttcgtttaacgtatttgagtattaccgattgagtaggattttgattgccaagctttttccttgccatatatccgtcctttcaatcgtatcgcatgataaccctatcgctgggagaaattagatcaccgaccctttctcttgtaccaaggtttctgcagttgcttaacctgctcctgcaattcagtaatcaaaagcccctgttgctcgaacagctgattgattgactggacattcttcgaaccatcttcctcgaaacgctccatccgctgaacaacttcttccacaataccagccaaaccgtcaagacttgctgaagtagccaaggcaagagtagcaactgccgccaaccgcacctcctggtcacgctttttcttaatacgcttattcatagcgttctcctttctatttttttgcaacaaaaaagcactcgattactcgaatgcttatccccacccaaacgacaactctttacgatagttcccgtcttggtctttaaaattttgtaaaaacttccctaaatacttcgtgttggcatcgtcaaatgtattaacacgaatcactttagtttcagggttaaaaacaagcgtttgcttatgctcctccttgttttcagggtaaatctcatagtgataaaaaccatcgtcaccaatatctatcaaaacaaatcttaaatacataaacttaccctttctttttaaattcattgacagcttcggagtaattaaataattcatctacttttttgtgagctgtatcataatccaacttgctagtcacatacatataatcatgttctaagcgttcatgctttagcaaaattaaatcatgcggttgaatgtttttcccagaaattaatctctcccatgattgagccatgtcaatgtctggtgcaaacaacccctttccataattcaactcatgaacattattaaacacatgatcgaacgctttatcaacatcatcttttgaaaagccattcatttctttgaacttcccaatgttcgaatagattttacgtttttccaaatcttgattgctgttttttatcctgtcgtaggttaaataagcatgtttttcggctttaataaaatcctttggcaaaagctcgtcgtttgaatcatcacgaacataattctttgcgccagcttgaacaagtagtccgatgcctttcctttcctctattttagcattttttaaaggatcagaccaacgtttagagtgtgcattctgttttttgccgtttcctgggtgataatcaacagtacacctgcaccgattatgcctcctccaaacatcctttggaacatctggatagttataaaccccaacaacagccctacaccatttacaacaattaccactttcttttctgacaactttaggtgtcaaccctgccttggcatgaaaatccgcattcgctttgataatatcatccacaatcgactgcgtaaagttcacaatcggctcctgcaacaaccaagccaccttatcgaactcatcctcagacgacaaacgattgacaagaccatcaatcctgtcctgattgacatcaggtacctgcacctgtagaccaatcttggcagacctattcaaatcctcctgaacttgaccagcataaccactcacaagctcgaaattccgccctagcgtgtccatcagtagcctttgggctatattgtaatacattttaccgtctggcaacctatcgcccgtcacagacgaactgagagcctctgacagaatgtccccaacctccagagcaaaatcatttgctgtggcataggttgcttttttctttttcagttcagcaaaagcatttctgaccacttcactcttcccataagaaacttcaaacttgtcctgaacttcttttagcaagtctggtaaaatatcacgttccatgcattcttactcctcagtattatatttccgaagattagcaactctgttcttaaatttttcaaaaagtaatctactgttttcttccctctctccttcaatagtcatttggtttccgactcctgataaaacgttttcgactaccggtggtagtggattacctgctataccggttaaatcacgaatagtctctgcggtgatataccccggcaaagcctggttaattttgataacaccatcacctaacatggtcaatgtcgttgcatcagcctcaaacaacggctcccatttaggaacagtcttgacgaactgttctctcaaaaacggatactcatcacgcaagcaagcagcaacataagcgacattcagcaagccactacctaaacttcgttgagcttttcgacctgctaatcgcaaattttcatgactagctttgatagcttccaccgatgatggattgtccgacacaaaaccaaggtcatctaatgtcaatcccatttccccagcgaaaccggccgccgcagtccgtaactgctcggtaaacggactcatgctagctgttgtaaattgtccaatgctcggcacgtcaccatcgtcatccttagtgaattccaagagactagtcacagtcgctttccacttgtccataggttccgcatcttgactcgtacccaaaacatatttctgcgggaacgaatagaactcagccgtcacatcggcacgttccagcgttcgcttggcatacgactgatagtacatacctgcacgagtgattcgactacgaccaaacggacgaaccgcatctggcctatgaatgactggcaccagtaacggacgacctgttggattggcaatcggagtgaacgttccacccgaaatgtaaattgtttgctctgctgtaaaataagcctctaacttcggactaccattatcatctcgttgcaagaccgcatagccttcggtcaatagccctgtaattggatcaagtatacctgtcgcgttactggactcaatgacttgcagtcgcactttgtcctctaccttggtcaaatagacaaagctacacgatccaatcaaggacgacaagaccaccgagtcgaagaacacatctggattgttttgctgaaaaatatcattgacctggaattgatcgttgtcaaactcgcgaaagaccaatctgtccgctaagctatccacacctttcgcgcaccagcccaagacagaccgatactgctgacgaatgtttggcggaatcgtaattccgaaagagctatctgtgtgctgcatagcatactgcttatacctcatttccactcgtggcttgacctcgtttagctttcgtctgaggtatccgatacctttatattccaatctggtctcctttcaattttggcgcgagaaaaaatgtatagtgacggcgtgaagtgccgacgagaatagagggagggtggtaccccccctacgttttcaacctcgataatttgcccaatttatgctttgtggcaaattgcgatttccaatcactttaggattgcttgatacatcgttgaatagtttgtcagatttttgtctattacattgcctgtgcgttaattgcatgttagatatatctgatggatgtccaccttttgcaattggtatgatatggtctattgctgcactcataggatgaggagctttgatagacttatcaactggatgtccacatatcgcacaaatcgttgcagtttttaatattctttttttatttttatctgcctcagtcctgtgcggaccaagcttatccgctcgtatgctaaccatatcatcctttcgttttaaaaaagccacacgattgtgtgactaatgcatattaggtcttggtccgatatgcgattgaccagacctccgagtcaaggtccccgtaggattcacttactcttaaacgggaacaacaggattcgaacctgcatattaagcaaccatgcttcaaaaggaatcaaaccttattcgcaagacttcccctcttgcgactctaccaattgagctatgttcccaatttagataccgagttcgattttctaaggagactggcagtcaattgacaatgactgaaatgttaacgtttatctcttctcggtatcttgacaatactattttaacatctcgaaaaacgaataatctatacaaaaacttttgatttcttggtcaaaactctaattttgaactaacaaacttccgcttctatattgttccgcaaacgataacagtgcatcgttgagttcgatatagaaactagcttctgataagtacaagtcgttgtagatttcaaaatcataccgcttgcctgcgtaaagatacttctcataaagaatgcgtctgtgcgtcggattgagtagattgttgatcgcatactcgatagcttcaagttctgcttccgcatctactcgattgattgccaagcgttccacgggcctgctaggattgccattagcttgtctcggttcgaaagtgtacactgcagtaactttctgtccatctacatcgttcgctactctacgccagcgagggtactctttcagttttcgttttgcattagctttcgttttttcgaaatcaacttctggaaaaaaagtcatctgctcatacctttctgtgatataatagttttagagttttattcacatagtcagtacaagtgtgctggctttttttatttcccccacggctgtcgctgatggctataatacgggtacactagccgaattttccctctcggagctagcaccttaggctcgtaaggcttgacttgctcgtacagctcgtctattttatccaacatgcgttgtcgcggtggtcgtccgtctagccatttgtagacagatagagtcgtcacgtccatctcggtcgcaaattggcccctcgtccatcctgtcttttgtaggatgtatttgattttatctgctgtggtcattccaaatcctccaacgctacccatctaaattgtgggtactttttagcttcttcttgggtgcaacgacatgcataggctctaacgaataattctgaatccgtatcaagaatcctatgtttcccattcgttctgacaagaatagaatactttgtcatcctcggctctggcacatcgaccagtagcacacctagtttttcagtcatctaatacctccatagccagagcaataacttgccctgtttcctcgttaaaatacatgagcgttgtaccattttttaaatttttgatatcatttttagtcagcttgaccgtatgcacatcgtactcattatcttcaaaacgtattttagctacttctcgatttgtcattatttgaactccttaaattgaaaatcaatgactctaatatgaacaggatttatcacagtattcccaatgatagccgcatgtccgttgttcaattgggaaatcaccttttctcggatttttattgcttcatcatcggatttaactgtatatacagtttttgaataatctgtacaaactgttattttacacataacctacccctccaccaattccggattttcgtagatgttgccagatagaaaaaccgtacaattttccaaacagtcaaacaaactatcccaaacttcttttcctgttttgacatccaacagcttccaacaaccatctctgtacacaattctagcttttcccgtatcttcaaattcatcaaaataagtccaaaaaattacatcatcttcaaataactcatcttcagaaaatgtactaaacagccctgtggattgcatgagaacgacatcatcaagcgggaccgtcatatgagtcgttccccgcagatgcttgattttgactttctgacgttcgtagcttattgctaagacctcgctcgttctttgcatggccttatgccatgctctaaacttcggtactaccattcttcgcctctttctacacgttcgaccaaacaatcgccacagtgacctgtctgaaagatactgccatagtccaatgtgccctctttatacttgcacccgcattcttcacaggtctcaattttcggtatcattttctgcctcctcgtttaaatactggatttttcttttcaagctctttctgcttatgatagtcattttctctccacatcactccatctggacctcttcggaccgacagcatgtaggaacttaaattttccatcactccacctctttcgcccactgccacagcctttcatcctgttgtttgatttcttgctctgttagtctagtagtataaatagccttgtcaaaaacatcagaaacatttccgaaaggcaacatcttatattgaacattactccctcttgtaattttcgtcagaatcgcctctgctatctccaccgtatacaacttctcctgctcgacctcatagccataaagataagctaagtgtagtttttttgcattgcaatcataattttcataaacccaattctgaacagattcgtcaacaacatcataattgagaccaaacagaccatgttcatctaatgtatctatccactccgccacaaacttcggcaccacaaccttctgcggttcgtgttgtttcaggaaccgatacacttgtttaaccatcattctaaaaggatgtgtggcgctatgcactgtgacaccaactccgtcagcgaatgtttcattgattgttacaggtataaactccgtacacccgttcacttcgtaaaaatcattctgtttcatctgtttccctctctacaaaaatagtgtgacaatccaaagtagaacgaatgtaataataggggttccgattacttttgccaacaattcggcaaaatcataatcagcttcctttcgtccaccgctaacaataatatagctgaccagtatatcaagacctatcgcttgtgctagtgttatacttgggactccgtctaaagttgttaagatgttattccatccatattgtattaccaatcccgataagataaagcttaatgggaatacaaacaatactgccagtaattgttctcttgtgctcattttattattcatctgtttcctccttgaaaaaggtatcaaagtccaaccaatcatctttaataagatttccaatcttcgttactctacatccaaaaccgttactctcaatacggatatacttcccttgcagttcttcccagctatttgcaccgacaacctccaagatacggtctatcagctcaaagctcttgtaaaaagctactcgttttttcttgtgttcgtcgtatttatctaggcagtatcctccaatagatactccagatccatatccctcaacagtcagataacaagttaaaattccatggtcttctctgcccaagaaagttttggttatctttacgttttcaattgttttattcatctgttccctccaactcctcaatcaaccaatctagattctgacgagccttcttcaagtcctcaacaccgttcttctgctgaaatcgcaacagatacttgatgacattgccccaatagtaggcgcgctcgcctgctaaatcccagataaaattcttgaccacttccaaggcttccataccatacttcccttggtaatgttttggtttggttacgttgttaaattgttcttcttgtccctcacattcaggacaagtgcaagcgtaaatcatttgtgtcataaaatttcctccactttaatctcaattctcggtctaggactgtacacctttcttgttgtatgctcgacaatgatgttatcatccgtccaaacacacccagcattgctgatgctgtcataaaccgctttttccagattatctagatctggttttttatctacgtaaattcgctcattgataaagtcatcatactgtcgcactttcttggccttggaccgaggcttaggcggttctgacatggacttcggagcaggcatgtagaatgtcatgtctacctttattggcccgtcgaaatatggcccgtcataccgttgtctaacaagttcagtacatttcttcctccaagcaaccatcttaccgtcttcgtaaaccgtcgcatgtctgccttgtacactagctctcggtctgctttgaggtttgggttcaattggtataatcaacctcataccagaactccataaaatcctaattcctcaaacaaattctttttatttccctcgataaactcaaacaaggtctgaatttcttttatgtcttttcctagttctttcgcaatattggaatcatcaaggaagagcttatctggatattcagcttctaaaaccagtctatattctggttcaaacatatctccattttcatccagagatatttggttatcttgcacaacaaactctgctttgatgttccataggctaccgccaatcacttccatactctgcttcacttcatttgtcacaagtgtgaaaggtgtttttaaaattgctgtttttttcattttttctttccaaaatccagcgactgccattgtgtgagtttggctaaatacgggcagtcgctgtcgtccaactgtcaactgattgtttccaattgacacgctttctagttcgcttttttcgtggttcacggcacgttagtttcatatttccaacaattcactaatcgacactatgcgattcagtttctttttgctacggcaataatcacattttccgcacttaatcggcccaactttcccagttgccacatcccatacatggctagcacgctcagaaacgaattctaagccctctgagagcatttcttcgtcaaggcttaatatttccttgtctggctcgttttccttgctcacagcgacgattagaggcctatacgaacgtcctgtcatttgcttcagcaattctcggtaaacagacaattgagcatggtagttgtagttaataatatttgccaatgcaccgggaactttttgtttcaagtcttgactccactctttatcataaattgttttcatagtcttcaaatcaaccatgtatcctcttgtgtggttgatagagtcaatcttgcctttaaatggcgtgcctgcaatagttcctgtcacaatcatctctttctctacctggtctccttttcgaccgtggtatagattattgaacagctcatcagtttccaaggttgcgataaccttatctgccaatttgtaatcagccagcatgccgtatggtttacgactggaaaacaaatctgacttatgctcttctaaaaatttctgatgagcttctgcactttcaaaatagctatggacataattaccaaaaatcaacggcttctggtctctgtcctctacccacttcccagtatccagagccaatgcttttgcctcgcattctagatacttcttaaagcgggataccgacatgtagtccttatccttgtaataattatcttgcgttagggtcttcatatttcaaatccaactccccgttgtcttttagctcgcttacctcttctaagatttcgcctgtttcagtatctacattcatcaagtcttcaagcaactgttggtctgcattgactggttcagcagtcttgatgtctcgtgtcgcattgtcgttctcactttcgtcattctgcattcctgtttgcatctctactgacaacggtccatagatacttagaatatgtttcaataccgtcttgcgtgccattgcatcaaaatctgttttccaaggtccgttgccgaaagttttagaaaacttcttgccgtgcgcttcaacttcagcttttgtccaataagtcaactttctaaaaccatttagtaaactgaagctcgcaaaatacccgataacctcatcttgtggttgagtaaaatcaagggtcagttcttcaaacaatgggtcataagatacaaactgactcttgtaaaccggtcctgcattgatgctcttatatttaccgcttcgttgcgccagttcaatcaaacctttataccccaactggaattgtacctgccctttatacggaactaggtaagcacgacctaggcttggttcaatcggcaaattcaagactgctgccttcatggctgatgtcatgatcgattcgtttgatgcagattttagcgatgcattgccttggataacagacaagatacttgttgcaaactgtacccctgcgcctttccaaacatcatcaaatgatttttgcacatttggtgcattgaagaaatttttgtgtgttactacttgatttgttgtcatttctttctccttacttttcttaataacctgtttggtttcgccattcttcttgtagttctttaatcatttgaagatgactcatcttcatgattcgtcttttgtcttgatggctaccatcccaaacattagcaagcatgtcgtcttgtagttgctgtttgagcttttttaaacataatcgcttcttcaatgttttctccttttcactattcatagcccatcgctacattgtctttccattcgtcataggctctgtcctcgtcttcgtctattttccagatgtcaactggtggttctggtggtgtgcttaaccatgtatcataatcaaacatcaaaactccacctttccgccaatttcagaccaaccagcccactcatctaacttcttctggatgatatggtgcttctgttgtaacaacagccccctgacctcatcacctatctggccatacttttcttcgtggtcagcaatcatttttaatttctcttgcatcatccctcctaaaacggtaattctcgtctgctttgcgcattatctggatacttaaagatattgttcatcgcacctttcataattcggctaacaagtgagcggtcatacaccttctgcatctgttcccctgtcaggttggtgttgatgatggttgtatcacgttcatccaaaatctgatagaggatattctgcttccagtcattcgcttccttggtctgtctgccaaatgtcgattccttgcctaggtcatccagaaagagatagtcagcttttgtaagcatgtcaatcatctgctgggcactagtcccgtctttgtagccaaacccctcttggattcgttgaaacatttttggcacggagatgaacaacacgctcttcggttcagagattgaccgccaatctatatttagcttcctagcgatactgatagacagatggctcttgccgataccgggcttaccttggataatagcgttccctttgccttggtgcttaaagtaaaattcattcaacctcagagcaaactgtttcgcttcttcctcgattcgatttgtaattgtgtaggtcttgtaagatgcgtctttcaactcttttggtatcatgctcttcttagcgaatatgtcgtacgaactagcccacgtttcagcttccaaggcttggcccacctctctaatctgttgttcgttcatcttttccctggtgcattcagggcaacaagtgagataccgtggcactgtttcattcttgaccatgaccttgttctttgttcgccatagatagacctgatgtttcaaacacatctcgtctatcacatcatggacttcttctattttcattcaaaatcctttcagtttttggtataattcagtcaatccacaaaggaaggaggggattgaatgaaagtgattcctaaattactcgagatgtacaaaacagtcgaagttgaaatgaaaactggttctggatacttggttaaaagtcagaccgaaattcctgatttctacatcgcgagcgaactttcggagtaccacaatcaatacgtaaacgagtcaactgtttatatcaatcaagatgatatttcatctgctcgtggtgtcgtcgatactttgttcgttgatagcgacgtctaagttatcgattaatctctttgaagtggtgtaaacaccccaaccatagtccgttaaagctttatcaatattctttccaaataatgattcaacagctgaaacggatttttttagtgtcttaataatttcttgttctttcatttgcacctctttctagtagggtggtgggtagtttggatctggcacccaatcgtttgatttttgtttatttgctttactatcgacaaactgtctctgctcctcgtcctgtcgggcgacggttctgatgtcgttttttgcccaattagttaagatagagttgatataattgaaatatcgcttagaaccatcagcagctttatcgatagccctcttgaccaattcgggctcaaaatgttcaatttcaagatactctttcaacttttcatattgaaaaccatctaaagaatcaatcctttgttgatagtagtcataaatatttgcgtcagtagcagtttttactgtatctacttctaactctatatttatatctatctctttctctatctctatatctccgttgcgttctgttgcatcggtgttgcattgcaacgctttttgcatctctcgatgcttgcgagacctacgggtgcttgcggtttcgctacctatcaactctggaacttgctcaagttgaaactcgaatttgtcctttgtggttagcaattttttatttgttaaaaacaacaaggtcattcttaccgcctcaacatcttcatctatcagcaaagcgatttcttctgcaaggtcattccctaggttttcaaaatagatttttccactatcttgtagactggctaacatgattttgagatagataatcgtatgttcgtcaccgcctggcaatcgtctgagaagtttcatttcttttgaagcaaagaaatcttctttcaactgcaaccagtaatatcttttgttttgagttaatgccatcttccctcgactttctaactacccactaatatctgataagcttctaaatccagctccaccatgactttcaaattcttggtagatgtctcgatttgtttgcggtatgctgcgatccctgcttgccgttcttcttctgtctgagcgatgaaatagccgaaatggttgtcacgttttagcccgtaaacggggattttgaacttgtttctcaacttaacgacaatttgttctacagttcttttctcaaccccggtcactcgagtaatatcttttcgaggttttgggttatcaaatgttgcatgtcttacctctgcgaaaacaatccgctcattatctgtcattctgtcgatcatcttctgaagctccgtcatattccaaaatcctttctacatcttcaaggtgctcgatatcgccagttcgaagaaatcggtcgtatgattgagcaattagttctaatctaactaagcttgtcacccttctagaaccctcctcaaccgttcgttttcatcccttaatcgctgattttcgatgcggtattcgttccgttgttcagcgatgtcacgaaccatatcgtgcaacagttgattttcttgctctagtgagtaaagcggtcgagagatagcaggtttttcttgttttaaaaaattagctaaccattcttgcatatctgttctcctctttatcaatctgttgggcatttcgcttcaggctattcttcatagactcttttcgacaagttttgtaatactgttcagcctgtgcccatgtcttatatctaagcttttcaagctctgtctcaaggagcaaggctatctccctgtgcttccgttccttctcagccttccgttgttccagaactgctaccgcaagcatcggcgctgcgaaaattcctaatgttaaaattgcttctgtcatgcactcaatcccttcactttctttttcactttcatctttgctttgtaatattcaatgtctctttggtcaaaacggaaatgagttccagccatgtgataagggattttcccagccctcacaattgccataaatgggttacggctcatccctatgatttcacaggcttccttcacaccgatcgctttattggagatttgagagttttttctctcgatagcaagttctttccgaacttcactcaagacttcttggataatctcttgtttcaagaccttgaacgcttctaacatagtatccatcttgtcaaacctcgctttcgtgtgttatacttcaagtaagtaattttagtaagagcctgattaccgtcaggctttttttgtttttcaagcaacatcatcaaccaaaaatttattgataaaatactgctgacctttgcctgtaacttttacagttttgctaattgagatatgaccgtcagcatgtgtgatagttgtctcttttatttcaaataaacctagttccatagacttctgcgttggcatgttccaatcactgcccttgcgcttaatgagatagccgttctcacgcaaccaagcaaacaagcgattggcaccgattttaaagccgttttggctgattaacttagctaggtctccaaccaagatagacgagtgactagcactcacagcgtctgcaaacagcaccttgggcttgtccgcttcaatctgtgcttccagctgatggacctttttgtctgccagtagcagagcgcgagccataatcttctctggactgttgaagtccttttcaacctggataaagtactgacgaacctgcttgccacggtctgtccgttggatcatagcaatttccttggccatgtccagcttgataatgtggtcaaccttgttgtgacctccgcgacctgtttgcttcacaaaattgttaagcaaaaaatcttgattttctgcaaagccatactcaaccattcggtcaaaccacatagagtatggtgttttgacccccaaagcctcatgtaactgccgaccagacacaacaggctcatggttgtcgtttagatttacgttaataatttcgtgcataatactcctttcaatatttattattcttcaaatttttcccacggctcacggatgcccaataattttgaaacacgcaatttcaaatcagcactccctttccctttggtcaacaaatctgtgattgtgccttgactacgtaatccgacagcttgtgtcaaatcagcttttgtccagcctttttcagccaatcgtttttccaccagttctatccatttttgatgttgttgactcataaacttctcctttctctttattagttagaaagtaaagcgaaagtttttgcgaaattttatagattccacttgactttttacaaactatagtctaaaatcaagacataagaaaaacaccgaacaaattaaccgataacactataattcaactcgccaaagttttatttttttagttttatcttcgttttttgtttcgctttattattcgctttacaaattatattctatactaaagtttgtatactgtcaactatttttacaaacttttttctagaatttttttcgtaatgcttagaaaggttgttaaatcaatgttctcaacgttcgaaagaataaaagaattagctaaatctagaggtgttacgctaggtggtctagaagaaagattggggttaagtcgaaattctatttataccatgaaaaacaaaaagccttcagctgagaggctacagttaatagccgactacttcaacgtatctactgattacttacttggtcggactgataatccaagagttgctaaaactgatgacgaaattgataaaatagatttcaaagagctagcagccgagtcaatgtcttatgacggcaagccgtttgacgaagacgatatagagtttttctcatatatcatggaacagcactttaagaacaaatataaggaatagtaaaatgaccgcattagacctttgcgtacagcaaggtattgatattttattctttgatggtagagaaagagataaaaaagccttcttcaacaaacgtgctaatcttgttggaatagacacgtatgtagatggtatcgaacgagataagctactctatcacgaacttggtcataaaaaccatacaccctatcaataccaattacatagagagctgtgcgaacttcaagcaaacaggaatatgattcatcatttgctgaaagatgaattgtcgatgttagacgattataacgactttaactatgttcgttttatggaacgacatggcttaaaaactatgaccgatgaaagcatggttattgaagaatttcgcaccctaactggaaaacatttataataaggagaacatccaatgaagaacaacaccaacactttgcccttttatttaagaggttggtttttcttgatactacttatactatctatcccaacttacttatcgtcgttaatcctcttaataggattgttcttgattaggaataaaaaatatcccaatctctctcccgaccaacaagccagatggaacgaaattcttttagcgaatgaacaagctgataacatactaaaaactgcaaaggaagaagctgataatctaataaacaatgctaaaaaagaagctaaagactccattgatatggctaatacaattgtcgcaggagtggaatcgaaaaagaataatctcaaagaagagattgataaactagaaatagctaaaaaagaggctgagctttatttatcagaaaaagccgatgcattactttttaaagaaacaacggtagatttcacagacaatataacagctaatgaaatcaaaaatgaattatctttaattcaattaaaagaaaaagaactaataaaagctgatgttgcaataaataatcttggaatacaaacaacgaaagctaatcttaacaaacaatctagacaacttctccgtgcattcaatgctgaatcggactactatgtatctaacattacagcaaaaaatgtagatagctatcgtaataaattagcaaaatcatttgaaaatctaaatgcactatttgcagttgacggagtaaaaatcagtcatgaacttctcacgctaaagttaaagcaactagatgtcatgtataaatatcaaaaacaacttgaggtcgagcgtgaattattgaaagctcaaaaagaagaaatacgcgaacaacagaaagtcgaaaaagaaatccaacaagcgaaagctaagttggaaaaagaagaaagacagttccaaaacgagatgtctaaactattgaagtatcttaacagcgctaacaacgaggtcgaacaaaatatatacgctgataaaattaaagagcttgaggacaaaattaaggagcttgaaaaagataaagaagacgttctcaagcgtgaaagtaacacaagagctggatttgtttatatcatttccaatatagggtcattcggtcaaaatgtctacaaaataggtatgacaagaagattagaaccgatggaccgtatcaatgaattaagtagtgcttctgttccatttccatttgatgttcacgctctaatctttagcgaggatgctcctgctctagagaatacgcttcacaattatttcagagataaagaagtaaataaagtcaatccacgtaaagagttctttaaagttgacttgcaagaaatcaaagagcttgttcataaagaatataacaataccgtacattttactgatttagcagttgcggaacagtattatgaaagcataaaattaagttctgaataacaaataaaaaatccccacactctccgaaccatcggccagtcgtggatatttatagggagaaatgccccctgtggtaattgaggagtttaataatttaatttgacaattgaaacacatcatgatacaatagagacaatcgaagtgaatgctcccccctgggagccctaaagagctattgtgtccgcacagtagctctttttgattttgaggtaaatatgctgactaaaccattcaaaactattgatgaacagattgagattctaaaatccagaaacctcactttcctgcatgagccttcagccaaaagaatattggctactgttggttactacgaacttatcaacggttataaggatattggtattgagacaggtgagactttcaaagatggctttacatttgaacaactttttcatgtttttaatatggacaaagaaatccgctcagcagtcaatgccgcgatacttgaaatagaagctcatctaagaaccgccctatcctatactgtagccaaacactacaccgcagaccagaatatctatctgaacagagaaaactacgaaagaggggatgataaatttcaaacatctcaacgagacaagttattaaagaaatgtcataaaatcatcaacgatgattctcatccctataaacattaccgagaaaaacacaaaaacgttccgccttggattctcgtaaaaggaatgacatttgggaacttgatagcattctataaacttcaaaaaagccaagtgaaatcagaaatagtcagtgaattgacaggtatcccagttgaattagtttctgacgatttcaagtcccttatcattaacattctgtacttccttttagcttaccgaaatcgttgtgcacaccttggaagggtctttaattttgaaacaaccaaaaacaagattcattacaacaaattattccatgataggatgaagataaccgaatctgaatacaagcaaggaaaaggacaatttggtctcgcaacccttgtatcatccttatcttggttttcaacaactggagaagtttaccaggtagtcacaatactcaattttaaaatacaagaagctatcaacaactatcttaaactctacccagcagacaaagattttatctacaatcaattaggtggagatttgataccaatcatataatgaaggagcactcaataaaaaaattacttacaggaacaattactctactatctgttgtaacacttgtagcgtgttctcaatcaaataaaggta

(19) United States
(12) Patent Application Publication
(10) Pub. No.: US 2021/0161977 A1
(43) Pub. Date: Jun. 3, 2021

(54) NOVEL STREPTOCOCCUS SUIS BACTERIOPHAGE STR-SUP-1 AND USE THEREOF IN INHIBITING STREPTOCOCCUS SUIS BACTERIUM PROLIFERATION

(71) Applicant: Intron Biotechnology, Inc., Gyeonggi-do (KR)

(72) Inventors: Seong Jun Yoon, Seoul (KR); Soo Youn Jun, Seoul (KR); An Sung Kwon, Gyeonggi-do (KR); Eun Ji Lee, Gyeonggi-do (KR); Sang Hyeon Kang, Seoul (KR)

(21) Appl. No.: 15/734,586

(22) PCT Filed: May 31, 2019
(86) PCT No.: PCT/KR2019/006558

§ 371 Date: Dec. 3, 2020

(30) Foreign Application Priority Data
Jun. 4, 2018 (KR) 10-2018-0064062

(51) Int. Cl. A61K 35/76; A61P 31/04; A23L 33/10; A23K 10/16

(52) U.S. Cl. CPC A61K 35/76; A23K 10/16; A23L 33/10; A61P 31/04

(57) ABSTRACT

The present invention relates to a Siphoviridae bacteriophage Str-SUP-1 (Accession number: KCTC 13514BP) isolated from nature and characterized by having the ability to kill Streptococcus suis and having the genome represented by SEQ ID NO: 1, and a method for preventing and treating diseases caused by Streptococcus suis using the composition containing the Siphoviridae bacteriophage Str-SUP-1 as an active ingredient.

### TECHNICAL FIELD

The present invention relates to a bacteriophage isolated from nature, which infects Streptococcus suis to thus kill Streptococcus suis, and a method of preventing and treating diseases caused by Streptococcus suis using a composition containing the above bacteriophage as an active ingredient. More specifically, the present invention relates to a Siphoviridae bacteriophage Str-SUP-1 (Accession number: KCTC 13514BP) isolated from nature, which has the ability to kill Streptococcus suis and has the genome represented by SEQ ID NO: 1, and a method of preventing or treating diseases caused by Streptococcus suis using a composition containing, as an active ingredient, the bacteriophage described above.

### BACKGROUND ART

Streptococcus suis is a peanut-shaped gram-positive bacterium, and Streptococcus suis infection is known to be an important zoonotic disease that occurs worldwide. Streptococcus suis bacteria are classified into 29 serotypes depending on capsular antigens (Capsular, K).

Based on serotype reports of Streptococcus suis bacteria around the world, serotypes 1 to 9 have a large distribution, accounting for about 75% of the total thereof, and in most countries, it is known that serotype 2 is the most commonly isolated from diseased pigs.

Meanwhile, pigs infected with Streptococcus suis mainly show symptoms of anorexia, lethargy, rash, fever, and paralysis. In particular, respiratory infections such as pneumonia and the like may occur in finishing pigs, thus causing serious economic loss to the pig farming industry. In addition, Streptococcus suis is a known major pathogen causing meningitis, sepsis, arthritis, endocarditis, and vaginitis in pigs, and outbreaks thereof have been reported worldwide, including Korea, North America, Europe and the like. Therefore, there is an urgent need to develop methods that may be used to prevent and treat infection with Streptococcus suis.

Although various antibiotics have been used for the prevention or treatment of diseases caused by Streptococcus suis, the incidence of bacteria resistant to such antibiotics is increasing these days, and thus the development of other methods besides antibiotics is urgently required.

Recently, the use of bacteriophages as a countermeasure against infectious bacterial diseases has attracted considerable attention. In particular, these bacteriophages are receiving great attention due to strong antibacterial activity against antibiotic-resistant bacteria.

Bacteriophages are very small microorganisms infecting bacteria, and are usually simply called “phages”. Once a bacteriophage infects a bacterium, the bacteriophage is proliferated inside the bacterial cell. After proliferation, the progeny of the bacteriophage destroy the bacterial cell wall and escape from the host bacteria, demonstrating that the bacteriophage has the ability to kill bacteria. The manner in which the bacteriophage infects bacteria is characterized by the very high specificity thereof, and thus the range of types of bacteriophages that infect a specific bacterium is limited. That is, a certain bacteriophage may infect only a specific bacterium, suggesting that a certain bacteriophage is capable of providing an antibacterial effect only for a specific bacterium. Due to this bacterial specificity of bacteriophages, the bacteriophage confers antibacterial effects only upon a target bacterium, but does not affect commensal bacteria in the environment or in the interiors of animals. Conventional antibiotics, which have been widely used for bacterial treatment, incidentally influence many other kinds of bacteria. This causes problems such as environmental pollution and the disturbance of normal flora in animals. In contrast, the use of bacteriophages does not disturb normal flora in animals, because the target bacterium is selectively killed by use of bacteriophages. Hence, bacteriophages may be utilized safely, which thus greatly lessens the probability of adverse effects of use thereof compared to antibiotics.

Bacteriophages were first discovered by the English bacteriologist Twort in 1915 when he noticed that Micrococcus colonies softened and became transparent due to something unknown. In 1917, the French bacteriologist d'Herelle discovered that Shigella dysenteriae in a filtrate of dysentery patient feces was destroyed by something, and further studied this phenomenon. As a result, he independently identified bacteriophages, and named them bacteriophages, which means “eater of bacteria”. Since then, bacteriophages acting against such pathogenic bacteria as Shigella, Streptococcus Typhi, and Vibrio cholerae have been continually identified.

Owing to the unique ability of bacteriophages to kill bacteria, bacteriophages have attracted attention as a potentially effective countermeasure against bacterial infection since their discovery, and a lot of research related thereto has been conducted. However, since penicillin was discovered by Fleming, studies on bacteriophages have continued only in some Eastern European countries and the former Soviet Union, because the spread of antibiotics was generalized. Since 2000, the limitations of conventional antibiotics have become apparent due to the increase in antibiotic-resistant bacteria, and the possibility of developing bacteriophages as a substitute for conventional antibiotics has been highlighted, and thus bacteriophages are again attracting attention as antibacterial agents.

As described above, bacteriophages tend to be highly specific for target bacteria. Because of the high specificity of bacteriophages to bacteria, bacteriophages frequently exhibit an antibacterial effect only for certain strains of bacteria, even within the same species. In addition, the antibacterial strength of bacteriophages may vary depending on the target bacterial strain. Therefore, it is necessary to collect many kinds of bacteriophages that are useful in order to effectively control specific bacteria. Hence, in order to develop an effective bacteriophage utilization method for controlling Streptococcus suis, many kinds of bacteriophages that exhibit antibacterial effects against Streptococcus suis must be acquired. Furthermore, the resulting bacteriophages need to be screened as to whether or not they are superior to others in view of the aspects of antibacterial strength and spectrum.

### Technical Problem

Therefore, the present inventors endeavored to develop a composition applicable for the prevention and treatment of diseases caused by Streptococcus suis using a bacteriophage that is isolated from nature and is capable of killing Streptococcus suis, and further to establish a method of preventing and treating diseases caused by Streptococcus suis using the composition. As a result, the present inventors isolated a bacteriophage suitable for this purpose from nature and determined the sequence of the genome, which distinguishes the isolated bacteriophage from other bacteriophages. Then, the present inventors developed a composition containing the bacteriophage as an active ingredient, and ascertained that this composition is capable of being effectively used to prevent and treat diseases caused by Streptococcus suis, thus culminating in the present invention.

Accordingly, an object of the present invention is to provide a Siphoviridae bacteriophage Str-SUP-1 (Accession number: KCTC 13514BP) isolated from nature, which has the ability to specifically kill Streptococcus suis and has the genome represented by SEQ ID NO: 1.

Another object of the present invention is to provide a composition applicable for preventing or treating diseases caused by Streptococcus suis, which contains, as an active ingredient, an isolated bacteriophage Str-SUP-1 (Accession number: KCTC 13514BP), infecting Streptococcus suis, to thus kill Streptococcus suis.

Still another object of the present invention is to provide a method of preventing and treating diseases caused by Streptococcus suis using the composition applicable for preventing and treating diseases caused by Streptococcus suis, which contains, as an active ingredient, the isolated bacteriophage Str-SUP-1 (Accession number: KCTC 13514BP), infecting Streptococcus suis, to thus kill Streptococcus suis.

Yet another object of the present invention is to provide a disinfectant for preventing and treating diseases caused by Streptococcus suis using the said composition.

A further object of the present invention is to provide a drinking-water additive effective for farming management by preventing and treating diseases caused by Streptococcus suis using the said composition.

Still a further object of the present invention is to provide a feed additive effective for farming management by preventing and treating diseases caused by Streptococcus suis using the said composition.

### Technical Solution

The present invention provides a Siphoviridae bacteriophage Str-SUP-1 (Accession number: KCTC 13514BP) isolated from nature, which has the ability to specifically kill Streptococcus suis and has the genome represented by SEQ ID NO: 1, and a method of preventing and treating diseases caused by Streptococcus suis using a composition containing the same as an active ingredient.

The bacteriophage Str-SUP-1 was isolated by the present inventors and then deposited at Korean Collection for Type Cultures, Korea Research Institute of Bioscience and Biotechnology on Apr. 24, 2018 (Accession number: KCTC 13514BP).

In addition, the present invention provides a disinfectant, a drinking-water additive, and a feed additive applicable for the prevention and treatment of diseases caused by Streptococcus suis, which contain the bacteriophage Str-SUP-1 as an active ingredient.

Since the bacteriophage Str-SUP-1 contained in the composition of the present invention kills Streptococcus suis effectively, it is effective in the prevention (prevention of infection) or treatment (treatment of infection) of diseases caused by Streptococcus suis. Therefore, the composition of the present invention is capable of being utilized for the prevention and treatment of diseases caused by Streptococcus suis.

As used herein, the terms “prevention” and “prevent” refer to (i) prevention of Streptococcus suis infection and (ii) inhibition of the development of diseases caused by a Streptococcus suis infection.

As used herein, the terms “treatment” and “treat” refer to all actions that (i) suppress diseases caused by Streptococcus suis and (ii) alleviate the pathological condition of diseases caused by Streptococcus suis.

As used herein, the terms “isolate”, “isolating”, and “isolated” refer to actions that isolate bacteriophages from nature by using various experimental techniques and that secure characteristics that can distinguish the bacteriophage of the present invention from others, and further include the action of proliferating the bacteriophage of the present invention using bioengineering techniques so that the bacteriophage is industrially applicable.

The pharmaceutically acceptable carrier included in the composition of the present invention is one that is generally used for the preparation of a pharmaceutical formulation, and examples thereof include lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methylcellulose, methylhydroxybenzoate, propylhydroxybenzoate, talc, magnesium stearate, and mineral oil, but are not limited thereto. The composition of the present invention may further include lubricants, wetting agents, sweeteners, flavors, emulsifiers, suspension agents, and preservatives, in addition to the above components.

The bacteriophage Str-SUP-1 is contained as an active ingredient in the composition of the present invention. The bacteriophage Str-SUP-1 is contained at a concentration of 1×10¹ pfu/ml to 1×10³⁰ pfu/ml or 1×10¹ pfu/g to 1×10³⁰ pfu/g, and preferably at a concentration of 1×10⁴ pfu/ml to 1×10′⁵ pfu/ml or 1×10⁴ pfu/g to 1×10′⁵ pfu/g.

The composition of the present invention may be formulated using a pharmaceutically acceptable carrier and/or excipient in accordance with a method that may be easily carried out by those skilled in the art to which the present invention belongs, in order to prepare the same in a unit dosage form or insert the same into a multiple-dose container. Here, the formulation may be provided in the form of a solution, a suspension, or an emulsion in an oil or aqueous medium, or in the form of an extract, a powder, a granule, a tablet, or a capsule, and may additionally contain a dispersant or a stabilizer.

The composition of the present invention may be prepared as a disinfectant or a drinking-water additive or a feed additive depending on the purpose of use thereof, without limitation thereto. In order to improve the effectiveness thereof, bacteriophages that confer antibacterial activity against other bacterial species may be further included in the composition of the present invention. In addition, other types of bacteriophages that have antibacterial activity against Streptococcus suis may be further included in the composition of the present invention.

These bacteriophages may be combined appropriately so as to maximize the antibacterial effects thereof, because their respective antibacterial activities against Streptococcus suis may vary from the aspects of antibacterial strength or spectrum.

### Advantageous Effects

According to the present invention, the method of preventing and treating diseases caused by Streptococcus suis using the composition containing the bacteriophage Str-SUP-1 as an active ingredient provides the advantage of very high specificity for Streptococcus suis compared to conventional methods based on existing antibiotics. This means that the composition can be used for preventing and treating diseases caused by Streptococcus suis without affecting other useful commensal bacteria, and has fewer side effects attributable to the use thereof. Typically, when antibiotics are used, commensal bacteria are also harmed, ultimately lowering the immunity of animals and thus causing various side effects owing to the use thereof. Meanwhile, in the case of various bacteriophages exhibiting antibacterial activity against the same bacterial species, the antibacterial effects of the bacteriophages are different with regard to antibacterial strength or spectrum [the spectrum of the antibacterial activity of the bacteriophages applied to individual bacteria strains in terms of the various strains of bacteria belonging to Streptococcus suis, bacteriophages usually being effective only on some bacterial strains, even within the same species, and the antibacterial activity of bacteriophages thus depending on the bacterial strain even for the same species of bacteria]. Accordingly, the present invention can provide antibacterial activity against Streptococcus suis discriminating from that of other bacteriophages acting on Streptococcus suis. This provides a great difference in effectiveness when application to industrial fields.

### DESCRIPTION OF DRAWINGS

FIG. 1 is an electron micrograph showing the morphology of the bacteriophage Str-SUP-1.

FIG. 2 is a schematic diagram showing the difference in genetic characteristics by comparing the genome sequences of the bacteriophage Str-SUP-1 and the Streptococcus bacteriophage phi5218 having relatively high genome sequence homology thereto.

FIG. 3 is a photograph showing results of an experiment on the ability of the bacteriophage Str-SUP-1 to kill Streptococcus suis. Based on the center line of the plate culture medium, only the buffer containing no bacteriophage Str-SUP-1 is spotted on the left side thereof and a solution containing the bacteriophage Str-SUP-1 is spotted on the right side thereof. The clear zone observed on the right side is a plaque formed by lysis of the target bacteria due to the action of the bacteriophage Str-SUP-1.

### MODE FOR INVENTION

A better understanding of the present invention will be given through the following examples, which are merely set forth to illustrate the present invention, and are not to be construed as limiting the scope of the present invention.

### EXAMPLE 1

### Isolation of Bacteriophage Capable of Killing Streptococcus suis

Samples collected from nature were used to isolate a bacteriophage capable of killing Streptococcus suis. Here, the Streptococcus suis strains used for the bacteriophage isolation were obtained from the Korean Collection for Type Cultures (Accession number: KCTC 3557).

The procedure for isolating the bacteriophage is described in detail herein below. The collected sample was added to a THB (Todd Hewitt Broth) medium (heart infusion, 3.1 g/L; peptone, 20 g/L; dextrose, 2 g/L; sodium chloride, 2 g/L; disodium phosphate, 0.4 g/L; sodium carbonate, 2.5 g/L) inoculated with Streptococcus suis at a ratio of 1/1,000, followed by shaking culture at 37° C. for 3 to 4 hr. After completion of culture, centrifugation was performed at 8,000 rpm for 20 min and the supernatant was recovered. The recovered supernatant was inoculated with Streptococcus suis at a ratio of 1/1000, followed by shaking culture at 37° C. for 3 to 4 hr. When the bacteriophage was included in the sample, the above procedure was repeated a total of 5 times in order to sufficiently increase the number (titer) of bacteriophages. After the procedure was repeated 5 times, the culture broth was centrifuged at 8,000 rpm for 20 min. After centrifugation, the recovered supernatant was filtered using a 0.45 μm filter. The filtrate thus obtained was used in a typical spot assay for evaluating whether or not a bacteriophage capable of killing Streptococcus suis was included therein.

The spot assay was performed as follows. A THB medium was inoculated with Streptococcus suis at a ratio of 1/1,000, followed by shaking culture at 37° C. overnight. 3 ml (OD₆₀₀ of 1.5) of the Streptococcus suis culture solution prepared as described above was spread on a THA (Todd Hewitt Agar: heart infusion, 3.1 g/L; peptone, 20 g/L; dextrose, 2 g/L; sodium chloride, 2 g/L; disodium phosphate, 0.4 g/L; sodium carbonate, 2.5 g/L; agar, 15 g/L) plate. The plate was left on a clean bench for about 30 min to dry the spread solution. After drying, 10 μl of the filtrate prepared as described above was spotted onto the plate which Streptococcus suis was spread, and then left for about 30 min to dry. After drying, the plate that was subjected to spotting was standing-culture at 37° C. for one day, and then examined for the formation of a clear zone at the position at which the filtrate was dropped. In the case in which the filtrate generated a clear zone, it was judged that a bacteriophage capable of killing Streptococcus suis was included therein. Through the above examination, it was possible to obtain a filtrate containing a bacteriophage having the ability to kill Streptococcus suis.

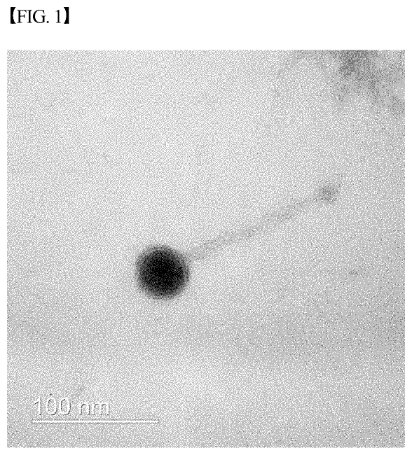

The pure bacteriophage was isolated from the filtrate confirmed to have the bacteriophage capable of killing Streptococcus suis. A typical plaque assay was used to isolate the pure bacteriophage. Specifically, a plaque formed in the course of the plaque assay was recovered using a sterilized tip, added to the Streptococcus suis culture broth, and then cultured at 37° C. for 4 to 5 hr. Thereafter, centrifugation was performed at 8,000 rpm for 20 min to obtain a supernatant. The culture broth of Streptococcus suis was added to the obtained supernatant at a volume ratio of 1/50 and then cultured at 37° C. for 4 to 5 hr. In order to increase the number of bacteriophages, the above procedure was repeated at least 5 times, after which centrifugation was performed at 8,000 rpm for 20 min to obtain a final supernatant. A plaque assay was performed again using the final supernatant thus obtained. In general, isolation of a pure bacteriophage is not completed when the above procedure was performed once, so the procedure was repeated using the plaque formed as described above. After at least 5 repetitions of the procedure, the solution containing the pure bacteriophage was obtained. The procedure for isolation of the pure bacteriophage was repeated until the generated plaques became generally similar to each other with regard to size and morphology. Additionally, final isolation of the pure bacteriophage was confirmed using electron microscopy. The above procedure was repeated until isolation of the pure bacteriophage was confirmed using electron microscopy. The electron microscopy was performed according to a typical method. Briefly, the solution containing the pure bacteriophage was loaded on a copper grid, followed by negative staining with 2% uranyl acetate and drying. The morphology thereof was then observed using a transmission electron microscope. The electron micrograph of the pure bacteriophage that was isolated is shown in FIG. 1. Based on the morphological characteristics thereof, the novel bacteriophage that was isolated above was confirmed to belong to the Siphoviridae bacteriophage.

The solution containing the pure bacteriophage confirmed above was subjected to the following purification process. The solution containing the pure bacteriophage was added with the Streptococcus suis culture broth at a volume ratio of 1/50, based on the total volume of the bacteriophage solution, and then further cultured for 4 to 5 hr. Thereafter, centrifugation was performed at 8,000 rpm for 20 min to obtain a supernatant. This procedure was repeated a total of 5 times in order to obtain a solution containing a sufficient number of bacteriophages. The supernatant obtained from the final centrifugation was filtered using a 0.45 pm filter, followed by a typical polyethylene glycol (PEG) precipitation process. Specifically, PEG and NaCl was added to 100 ml of the filtrate reaching 10% PEG 8000 and 0.5 M NaCl, which was then allowed to stand at 4° C. for 2 to 3 hr. Thereafter, centrifugation was performed at 8,000 rpm for 30 min to obtain a bacteriophage precipitate. The resulting bacteriophage precipitate was suspended in 5 ml of a buffer (10 mM Tris-HCl, 10 mM MgSO4, 0.1% gelatin, pH 8.0). The resulting material may be referred to as a bacteriophage suspension or bacteriophage solution.

The bacteriophage purified as described above was collected, was named bacteriophage Str-SUP-1, and was then deposited at the Korean Collection for Type Cultures, Korea Research Institute of Bioscience and Biotechnology on April 24, 2018 (Accession number: KCTC 13514BP).

### EXAMPLE 2

### Separation and Sequence Analysis of Genome of Bacteriophage Str-SUP-1

The genome of the bacteriophage Str-SUP-1 was separated as follows. The genome was separated from the bacteriophage suspension obtained using the same method as described in Example 1. First, in order to remove DNA and RNA of Streptococcus suis included in the suspension, 200 U of each of DNase I and RNase A was added to 10 ml of the bacteriophage suspension and then allowed to stand at 37° C. for 30 min. After being allowed to stand for 30 min, in order to inactivate the DNase I and RNase A activity, 500 μl of 0.5 M ethylenediaminetetraacetic acid (EDTA) was added thereto, and the resulting mixture was then allowed to stand for 10 min. In addition, the resulting mixture was further allowed to stand at 65° C. for 10 min, and 100 μl of proteinase K (20 mg/ml) was then added thereto to break the outer wall of the bacteriophage, followed by reacting at 37° C. for 20 min. Thereafter, 500 μl of 10% sodium dodecyl sulfate (SDS) was added thereto, followed by reacting at 65° C. for 1 hr. After reaction for 1 hr, the resulting reaction solution was added with 10 ml of a mixed solution of phenol, chloroform and isoamyl alcohol at a component ratio of 25:24:1 and then thoroughly mixed. The resulting mixture was then centrifuged at 13,000 rpm for 15 min to thus separate layers thereof. Among the separated layers, the upper layer was selected, added with isopropyl alcohol at a volume ratio of 1.5, and centrifuged at 13,000 rpm for 10 min in order to precipitate the genome. After collecting the precipitate, 70% ethanol was added to the precipitate, centrifuged at 13,000 rpm for 10 min to wash the precipitate. The washed precipitate was recovered, vacuum-dried and then dissolved in 100 μl of water. This procedure was repeated to thus obtain a sufficient amount of the genome of the bacteriophage Str-SUP-1.

Information on the sequence of the genome of the bacteriophage Str-SUP-1 thus obtained was subjected by performing next-generation sequencing analysis using an Illumina Mi-Seq sequencer provided by Macrogen. The finally analyzed genome of the bacteriophage Str-SUP-1 had a size of 33,991 bp, and the whole genome sequence is represented by SEQ ID NO: 1.

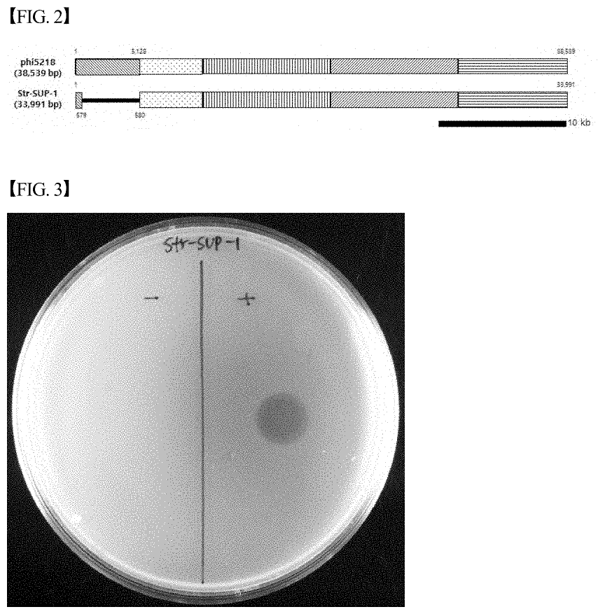

The homology (similarity) of the bacteriophage Str-SUP-1 genomic sequence obtained above with previously reported bacteriophage genomic sequences was investigated using BLAST on the web. Based on the results of BLAST investigation, the genomic sequence of the bacteriophage Str-SUP-1 was found to have relatively high homology (identity: 99%) with the sequence of the Streptococcus bacteriophage phi5218 (GenBank Accession number: KC348600.1). However, the bacteriophage Str-SUP-1 has morphological features of Siphoviridae and the Streptococcus bacteriophage phi5218 has morphological features of Podoviridae, between which there are obvious morphological differences. Furthermore, the number of open reading frames (ORFs) on the bacteriophage Str-SUP-1 genome was 56, whereas the Streptococcus bacteriophage phi5218 was found to have 64 open reading frames, based on which these two bacteriophages were evaluated to be genetically different. The difference in morphological and genetic characteristics between these two bacteriophages can indicate that there are external and functional differences in various characteristics expressed in various ways between the two bacteriophages. Moreover, the difference between these two bacteriophages also implies that there is a difference in industrial applicability of the two bacteriophages. Meanwhile, the differences in genetic characteristics observed by comparing the genome sequences of the two bacteriophages are schematically shown in FIG. 2.

Therefore, it can be concluded that the bacteriophage Str-SUP-1 is a novel bacteriophage different from previously reported bacteriophages. Moreover, since the antibacterial strength and spectrum of bacteriophages typically vary depending on the type of bacteriophage, it is considered that the bacteriophage Str-SUP-1 can provide antibacterial activity different from that of any other previously reported bacteriophage.

### EXAMPLE 3

### Evaluation of Killing Ability of Bacteriophage Str-SUP-1 for Streptococcus suis

The killing ability of the isolated bacteriophage Str-SUP-1 for Streptococcus suis was evaluated. In order to evaluate the killing ability thereof, the formation of clear zones was observed using a spot assay in the same manner as described in Example 1. A total of 10 strains that had been isolated and identified as Streptococcus suis by the present inventors or obtained from the KCTC or Korea Veterinary Culture Collection were used as Streptococcus suis strains for evaluation of killing ability. The bacteriophage Str-SUP-1 had the ability to kill a total of 8 strains, including KCTC 3557, among 10 strains of Streptococcus suis, which was the experimental target. The representative experimental result is shown in FIG. 3. Meanwhile, the ability of the bacteriophage Str-SUP-1 to kill Bordetella bronchiseptica, Enterococcus faecalis, Enterococcus faecium, Streptococcus mitis, Streptococcus uberis and Pseudomonas aeruginosa was also examined. Consequently, the bacteriophage Str-SUP-1 did not have the ability to kill these microorganisms.

Therefore, it can be concluded that the bacteriophage Str-SUP-1 has strong ability to kill

Streptococcus suis and can exhibit antibacterial effects against many Streptococcus suis strains, indicating that the bacteriophage Str-SUP-1 can be used as an active ingredient of a composition for preventing and treating diseases caused by Streptococcus suis.

### EXAMPLE 4

### Experiment for Prevention of Streptococcus suis infection using bacteriophage Str-SUP-1

100 μl of a bacteriophage Str-SUP-1 solution at a concentration of 1×10⁸ pfu/ml was added to a tube containing 9 ml of a THB medium. To another tube containing 9 ml of a THB medium, only the same amount of THB medium was further added. A culture broth of Streptococcus suis was then added to each tube so that absorbance reached about 0.5 at 600 nm. After the addition of Streptococcus suis, the tubes were transferred to an incubator at 37° C., followed by shaking culture, during which the growth state of Streptococcus suis was observed. As shown in Table 1 below, it was observed that the growth of Streptococcus suis was inhibited in the tube to which the bacteriophage Str-SUP-1 solution was added, whereas the growth of Streptococcus suis was not inhibited in the tube to which the bacteriophage solution was not added.

TABLE 1
Growth inhibition of Streptococcus suis

| | OD₆₀₀ absorbance value | | |
|---|---|---|---|
| | 0 min | 60 min | 120 min |
| | after | after | after |
| Classification | culture | culture | culture |
| Not added with bacteriophage | 0.506 | 0.862 | 1.395 |
| solution | | | |
| Added with bacteriophage | 0.506 | 0.281 | 0.135 |
| solution | | | |

The above results show that the bacteriophage Str-SUP-1 of the present invention not only inhibits the growth of Streptococcus suis but also has the ability to kill Streptococcus suis. Therefore, it is concluded that the bacteriophage Str-SUP-1 can be used as an active ingredient in a composition for preventing diseases caused by Streptococcus suis.

### EXAMPLE 5

### Animal Testing for Prevention of Disease caused by Streptococcus suis using Bacteriophage Str-SUP-1

The preventive effect of the bacteriophage Str-SUP-1 on diseases caused by Streptococcus suis was evaluated using weaned pigs. Ten 25-day-old weaned pigs were divided into a total of 2 groups (5 pigs per group) and reared separately in experimental pig-rearing rooms (1.1 m×1.0 m), and an experiment was performed for 14 days. The surrounding environment was controlled using a heater, and the temperature and humidity in the pig rooms were maintained constant, and the pig room floors were washed every day. A feed containing 1×10⁸ pfu/g of bacteriophage Str-SUP-1 was provided to pigs in the experimental group (administered with feed containing the bacteriophage) in a typical feeding manner starting from the beginning of the experiment to the end of the experiment. For comparison therewith, a feed having the same composition but not containing bacteriophage Str-SUP-1 was provided to pigs in a control group (administered with feed not containing the bacteriophage) in the same feeding manner starting from the beginning of the experiment to the end of the experiment. For two days from the 7th day after the start of the experiment, the feed was further added with 1×10⁸ cfu/g of Streptococcus suis and then provided twice a day to all of the pigs in the experimental group (administered with feed containing the bacteriophage) and the control group (administered with feed not containing the bacteriophage), thereby inducing infection with Streptococcus suis. The detected level of Streptococcus suis in the nasal secretion of all test animals was examined daily from the date of feeding with the feed containing Streptococcus suis (from the 7th day after the start of the experiment).

The detection of Streptococcus suis in the nasal secretion (nasal swab) was carried out as follows. The nasal secretion sample was spread on a blood agar plate and then cultured at 37° C. for 18 to 24 hr. Among the resulting colonies, colonies presumed to be Streptococcus suis were isolated. The colonies thus selected were used as samples and subjected to polymerase chain reaction (PCR) specific to Streptococcus suis, and thus whether or not the corresponding colonies were Streptococcus suis was finally confirmed. The results of bacterial detection are shown in Table 2 below.

TABLE 2
Results of detection of Streptococcus suis (mean)

| | Number of colonies of Streptococcus suis | | | | | | | |
|---|---|---|---|---|---|---|---|---|
| | detected per plate | | | | | | | |
| Classification | D 7 | D 8 | D 9 | D 10 | D 11 | D 12 | D 13 | D 14 |
| Control | 17 | 18 | 18 | 17 | 16 | 17 | 14 | 13 |
| group | | | | | | | | |
| (administered | | | | | | | | |
| with feed not | | | | | | | | |
| containing | | | | | | | | |
| bacteriophage) | | | | | | | | |
| Experimental | 15 | 9 | 5 | 3 | 1 | 0 | 0 | 0 |
| group | | | | | | | | |
| (administered | | | | | | | | |
| with feed | | | | | | | | |
| containing | | | | | | | | |
| bacteriophage) | | | | | | | | |

As is apparent from the above results, it can be confirmed that the bacteriophage Str-SUP-1 of the present invention was very effective in the prevention of diseases caused by Streptococcus suis.

### EXAMPLE 6

### Treatment of Disease caused by Streptococcus suis using Bacteriophage Str-SUP-1

The therapeutic effect of the bacteriophage Str-SUP-1 on diseases caused by Streptococcus suis was evaluated as follows. Eight 25-day-old weaned pigs were divided into a total of 2 groups and reared separately in experimental pig-rearing rooms (1.1 m×1.0 m), and an experiment was performed for 14 days. The surrounding environment was controlled using a heater, the temperature and humidity in the pig rooms were maintained constant, and the pig room floors were washed every day. On the 4th day from the start of the experiment, 5 ml of the Streptococcus suis solution (10⁹ cfu/ml) was sprayed into the nasal cavity of all pigs. The Streptococcus suis solution used for nasal administration was prepared as follows. After culturing Streptococcus suis bacteria at 37° C. for 18 hr using a THB medium, the cells thereof were isolated and were then suspended in physiological saline (pH 7.2) to adjust the concentration of the cells to 10⁹ cfu/ml. From the day after forced infection with Streptococcus suis bacteria, 10⁹ pfu of bacteriophage Str-SUP-1 was nasally administered to the pigs in the experimental group (the group administered with the bacteriophage solution) twice a day in the same manner as the administration of the Streptococcus suis solution. Pigs in the control group (the group not administered with the bacteriophage solution) did not undergo any treatment. Feed and drinking water were provided equally to both the control and experimental groups. From the 3rd day after the forced infection with Streptococcus suis (the 7th day from the start of the experiment), all test animals were examined for the development of atrophic rhinitis caused by Streptococcus suis bacteria. The investigation of atrophic rhinitis caused by

Streptococcus suis bacteria was conducted by measuring the amount of nasal secretion. The amount of nasal secretion was indicated by indexing the normal level as ‘0’, a slightly high level as ‘1’, and a severe level as ‘2’ based on observation by a tester. The results thereof are shown in Table 3 below.

TABLE 3
Results of investigation of nasal secretion (mean)

| Days | D 7 | D 8 | D 9 | D 10 | D 11 | D 12 | D 13 | D 14 |
|---|---|---|---|---|---|---|---|---|
| Control | 0.5 | 0.5 | 0.75 | 1.25 | 1.5 | 1.5 | 1.75 | 1.75 |
| group (not | | | | | | | | |
| administered | | | | | | | | |
| with | | | | | | | | |
| bacteriophage) | | | | | | | | |
| Experimental | 0.25 | 0.25 | 0.25 | 0 | 0 | 0 | 0 | 0 |
| group | | | | | | | | |
| (administered | | | | | | | | |
| with | | | | | | | | |
| bacteriophage) | | | | | | | | |

As is apparent from the above results, it can be confirmed that the bacteriophage Str-SUP-1 of the present invention was very effective in the treatment of diseases caused by Streptococcus suis.

### EXAMPLE 7

### Preparation of Feed Additive and Feed

A feed additive was prepared using a bacteriophage Str-SUP-1 solution so that bacteriophage Str-SUP-1 was contained in an amount of 1×10⁸ pfu per gram of the feed additive. The feed additive was prepared in a manner in which the bacteriophage solution was added with maltodextrin (50%, w/v) and then freeze-dried, followed by final pulverization into a fine powder. In the above preparation procedure, the drying process may be substituted as drying under reduced pressure, drying with heat, or drying at room temperature. In order to prepare a control for comparison, the feed additive not containing the bacteriophage was prepared using the buffer (10 mM Tris-HCl, 10 mM MgSO4, 0.1% gelatin, pH 8.0) used in the preparation of the bacteriophage solution, in lieu of the bacteriophage solution.

Each of the two kinds of feed additives thus prepared was mixed with a pig feed at a weight ratio of 1,000, thus finally preparing two kinds of feed.

### EXAMPLE 8

### Preparation of Drinking-Water Additive and Disinfectant

A drinking-water additive and a disinfectant were prepared in the same manner because they differ only in utilization and are the same in dosage form. The drinking-water additive (or disinfectant) was prepared using a bacteriophage Str-SUP-1 solution. In the method of preparing the drinking-water additive (or disinfectant), the bacteriophage Str-SUP-1 solution was added so that the bacteriophage Str-SUP-1 was contained in an amount of 1×10⁹ pfu per ml of the buffer used in the preparation of the bacteriophage solution, and mixing was sufficiently performed. In order to prepare a control for comparison, the buffer used in the preparation of the bacteriophage solution was used without change as a drinking-water additive (or disinfectant) not containing the bacteriophage.

Each of the two kinds of drinking-water additives (or disinfectants) thus prepared was diluted with water at a volume ratio of 1,000, thus obtaining a final drinking water or disinfectant.

### EXAMPLE 9

### Confirmation of Feeding Effect on Pig Farming

Whether the use of the feed, drinking water and disinfectant prepared in Examples 7 and 8 was effective for pig farming was evaluated. In particular, the present evaluation was focused on measuring the extent of weight gain. A total of sixty 25-day-old weaned pigs were divided into three groups, each including 20 pigs (group A: fed with the feed, group B: fed with the drinking water, and group C: treated with the disinfectant), and an experiment was performed for four weeks. Each group was divided into subgroups each including 10 pigs, and the subgroups were classified into a subgroup to which the bacteriophage Str-SUP-1 was applied (subgroup-\textcircled{1}) and a subgroup to which the bacteriophage was not applied (subgroup-\textcircled{2}). In the present experiment, the weaned pigs were raised separately in individual subgroups. The subgroups were classified and named as shown in Table 4 below.

TABLE 4
Subgroup classification and expression in pig-farming experiment

| | Subgroup classification and expression | |
|---|---|---|
| | Bacteriophage Str-SUP-1 is | Bacteriophage is |
| Application | applied | not applied |
| Group fed with feed | A-\textcircled{1} | A-\textcircled{2} |
| Group fed with drinking | B-\textcircled{1} | B-\textcircled{2} |
| water | | |
| Group treated with | C-\textcircled{1} | C-\textcircled{2} |
| disinfectant | | |

In the case of provision of the feed, the feed prepared in Example 7 was provided in a typical feeding manner, as shown in Table 4, and the drinking water prepared in Example 8 was provided in a typical feeding manner, as shown in Table 4. In the case of disinfection, the disinfection was carried out alternately with conventional disinfection 3 times a week. Disinfection using a typical disinfectant was not performed on the day on which the disinfectant of the present invention was sprayed. Based on the experimental results, the extent of weight gain was significantly superior in the groups added with the bacteriophage Str-SUP-1 compared to the groups not added with the bacteriophage Str-SUP-1 (Table 5). For reference, the separation rate of Streptococcus suis bacteria in the nasal secretions of the test animals was also investigated as in

Example 5. Streptococcus suis bacteria were detected in the nasal secretions of some animals in the groups not applied with the bacteriophage Str-SUP-1. On the other hand, in all animals in the groups applied with the bacteriophage Str-SUP-1, Streptococcus suis bacteria were not detected during the experimental period.

TABLE 5
Results of pig-farming experiment

| | Weight | |
|---|---|---|
| Group | gain (%) | Note |
| A-\textcircled{1} | 108 | |
| A-\textcircled{2} | 100 | Based on average weight gain of this group (100%) |
| | | Streptococcus suis bacteria were |
| | | detected in some individuals |
| B-\textcircled{1} | 106 | |
| B-\textcircled{2} | 100 | Streptococcus suis bacteria were |
| | | detected in some individuals |
| C-\textcircled{1} | 105 | |
| C-\textcircled{2} | 99 | Streptococcus suis bacteria were |
| | | detected in some individuals |

The above results indicate that the feeding of the feed and the drinking water prepared according to the present invention and the use of the disinfectant according to the present invention were effective for pig farming. Therefore, it is concluded that the composition of the present invention is effective when used in raising pigs.

While the present invention has been particularly shown and described with reference to exemplary embodiments thereof, those skilled in the art will appreciate that the specific description is only a preferred embodiment, and that the scope of the present invention is not limited thereto. It is therefore intended that the scope of the present invention be defined by the claims appended hereto and their equivalents.

[Accession Number]

Name of Depositary Authority: KCTC

Accession number: KCTC 13514BP

Accession date: 20180424

1. A Siphoviridae bacteriophage Str-SUP-1 (Accession number: KCTC 13514BP) isolated from nature, which has an ability to kill Streptococcus suis and has a genome represented by SEQ ID NO: 1.

2. A composition for preventing and treating a disease caused by Streptococcus suis, comprising the bacteriophage Str-SUP-1 (Accession number: KCTC 13514BP) of claim 1 as an active ingredient.

3. The composition of claim 2, wherein the composition is used to prepare a feed additive, a drinking-water additive or a disinfectant.

4. A method of preventing and treating a disease caused by Streptococcus suis, comprising:
spraying to an environment the composition comprising the bacteriophage Str-SUP-1 (Accession number: KCTC 13514BP) of claim 2 as the active ingredient.

5. The method of claim 4, wherein the composition is in a form of a disinfectant.

6. A method of preventing and treating a disease caused by Streptococcus suis, comprising:
administering to an animal other than a human the composition comprising the bacteriophage Str-SUP-1 (Accession number: KCTC 13514BP) of claim 2 as the active ingredient.

7. The method of claim 6, wherein the composition is in a form of a drinking-water additive or a feed additive.
